(19) United States
(12) Patent Application Publication
(10) Pub. No.: US 2012/0245613 A1
(43) Pub. Date: Sep. 27, 2012

(54) Packing Strip Probe and a Method of Using Thereof

(76) Inventor: John Stephen YEE, New Smyrna Beach, FL (US)

(21) Appl. No.: 13/430,666

(22) Filed: Mar. 26, 2012

Related U.S. Application Data
(60) Provisional application No. 61/466,996, filed on Mar. 24, 2011.

(51) Int. Cl. A61B 17/00

(52) U.S. Cl. 606/190

(57) ABSTRACT

A packing strip probe is used to medically treat a sub-dermal cavity that is created by an abscess, an ulcer, or a deep wound. The probe comprises a rod, a blunt tip, and a probing tip. The rod is the main structure of the probe, and the blunt tip and the probing tip are located on opposite ends of the rod. The blunt tip is used to open up the sub-dermal cavity and to gently debride the sub-dermal cavity. The probing tip is used to aggressively debride the sub-dermal cavity and guide a packing strip into the sub-dermal cavity. The probing tip comprises tines, slots, and a rounded end. The tines can be used to either aggressively debride the sub-dermal cavity or wrap and insert the packing strip into the sub-dermal cavity.

The current application claims a priority to the U.S. Provisional Patent application Ser. No. 61/466,996 filed on Mar. 24, 2011. The current application filed in U.S. Mar. 26, 2012 while Mar. 24, 2012 was on a weekend.

### FIELD OF THE INVENTION

The present invention relates generally a medical device, more particularly, to a device for treating deep tissue wounds that include subcutaneous abscesses or deep chronic wounds.

### BACKGROUND OF THE INVENTION

A variety of locations within the body of a patient are subject to developing an abscess and/or wound ulcers after the occurrence of infection/contamination, or decrease nutritional supply at such locations. A variety of suction and other devices are known for cutting and removing a wide range of materials from the body at locations other than abscesses. Such devices usually possess a structure which is particularly adapted to the location of interest. The present invention introduces a device designed to improve safety and efficiency for incision/drainage and wound debridement procedures when treating abscess or tunnel wounds for medical or dental purposes.

The blunt tip was designed to replace the traditional procedure with sharps (ie scissors, Addison hemostat, etc.). By using the blunt tip, the probability of penetrating soft tissue organs, lacerating nerve fibers and blood vessels, and creating an unnecessary larger wound is minimal. The probing tip was designed to grab onto and pack the packing strip into the wound with ease. Traditionally, this is done with a thin friable, wooden end of cotton swab. Sometimes, the tip can snap or leave a small splinter into the wound. Other times, it can be challenging to grab onto the packing strip with a blunt end of the wooden stick.

### BRIEF DESCRIPTION OF THE DRAWINGS

FIG. 1 is a perspective view of the present invention.

FIG. 2 is a side view of the present invention.

FIG. 3 is a front view of the present invention.

FIG. 4 illustrates the procedure of guiding the packing strip with the present invention.

FIG. 5 is a front perspective view of a different embodiment of the present invention.

FIG. 6 is a side view of the different embodiment of the present invention.

FIG. 7 is a back perspective view of the different embodiment of the present invention.

### DETAIL DESCRIPTIONS OF THE INVENTION

All illustrations of the drawings are for the purpose of describing selected versions of the present invention and are not intended to limit the scope of the present invention.

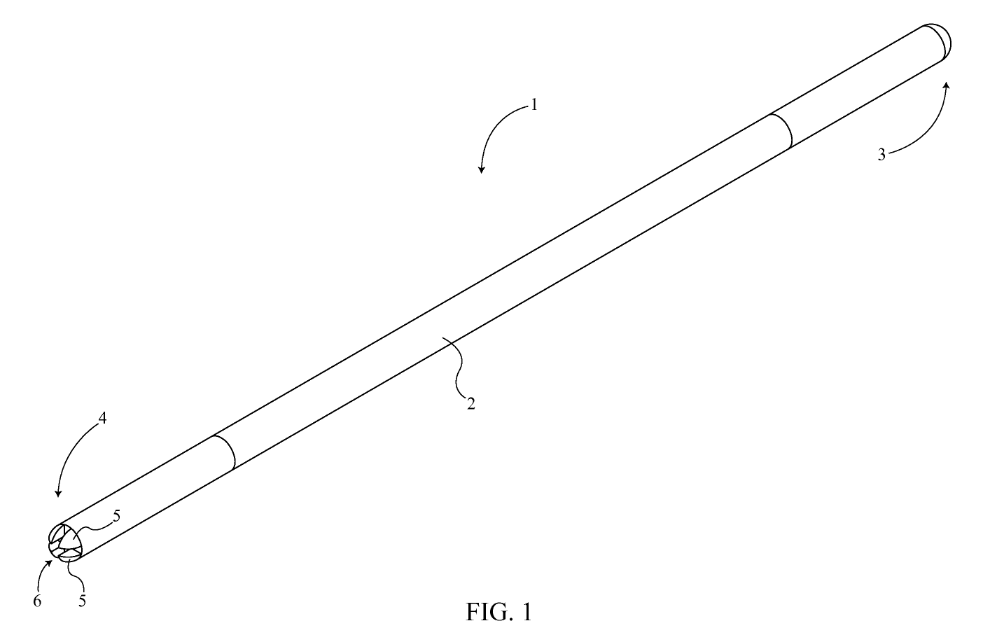

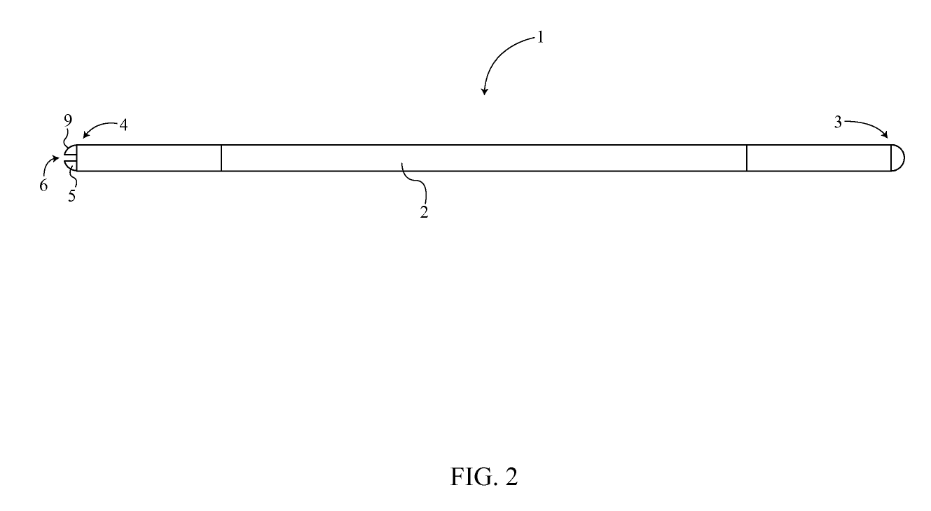

As can be seen in FIGS. 1 and 2, the present invention is a packing strip probe, which allows a user to medically treat an ailment that leaves a sub-dermal cavity such as an abscess, an ulcer, or a deep wound. The present invention comprises a rod 1, a blunt tip 3, and a probing tip 4. The rod 1 provides the present invention with its overall structure and allows user to extend the present invention into the sub-dermal cavity from a distance. In the preferred embodiment of the present invention, the rod 1 is solid and cylindrical and is made of either polypropylene, stainless steel, or durable plastic. Also in the preferred embodiment, the dimensions of the rod 1 can be ¼ of an inch in thickness and 8 inches in length. The blunt tip 3 is located at one end of the rod 1, and the probing tip 4 is located at the other end of the rod 1. The blunt tip 3 has a dome shape and is used to carry out tasks that are less invasive to the sub-dermal cavity. The probing tip 4 is used to carry out more invasive task such as guiding a packing strip into the abscess, the ulcer, or the deep wound in order to fill the sub-dermal cavity.

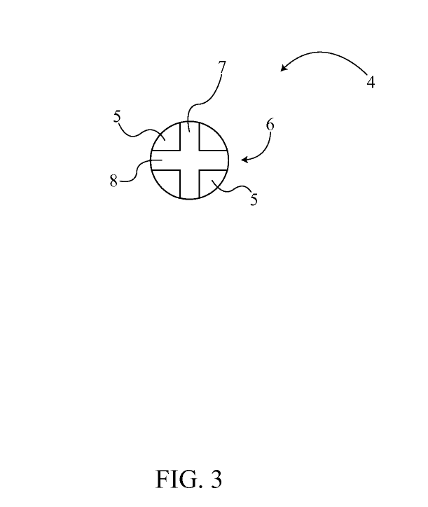

The present invention has a number of subcomponents that more specifically defines its functions. The rod 1 comprises a handle portion 2, which allows the user to maneuver the present invention while medically treating the sub-dermal cavity. The handle portion 2 is located in between the blunt tip 3 and the probing tip 4 so that the user can maneuver either the blunt tip 3 or the probing tip 4 with equal coordination. The probing tip 4 is more intricate than the blunt tip 3 and comprises a plurality of tines 5, a plurality of slots 6, and a rounded end 9, which are shown in FIG. 3. The slots 6 are cuts in the rod 1 that traverse into the end of the rod 1 and that laterally traverse through the rod 1. The tines 5 on the probing tip 4 are shaped by the positioning of the slots 6. In the preferred embodiment of the present invention, the plurality of slots 6 comprises a first slot 7 and a second slot 8, which are positioned perpendicularly to each other. The first slot 7 and the second slot 8 are the ideal number of slots 6 and shape the ideal number of tines 5, which is four. The rounded end 9 is located on the tines 5 opposite to rod 1 and is formed by the combination of all of the tines 5.

The process that the user follows to medically treat the sub-dermal cavity with the present invention occurs after making a surgical incision into the abscess, the ulcer, or the deep wound. The process of using the present invention begins by forcing the blunt tip 3 through the surgical incision and opening up the abscess, the ulcer, or the deep wound.

The blunt tip 3 is then used to gently debride the sub-dermal cavity of the abscess, the ulcer or the deep wound. The process proceeds by turning the present invention around and aggressively debriding the sub-dermal cavity with the plurality of tines 5 and the rounded end 9 on the probing tip 4. For example, debridement is done to remove the bioburden from the wound. Once debridement has been completed, the sub-dermal cavity can be irrigated with normal saline.

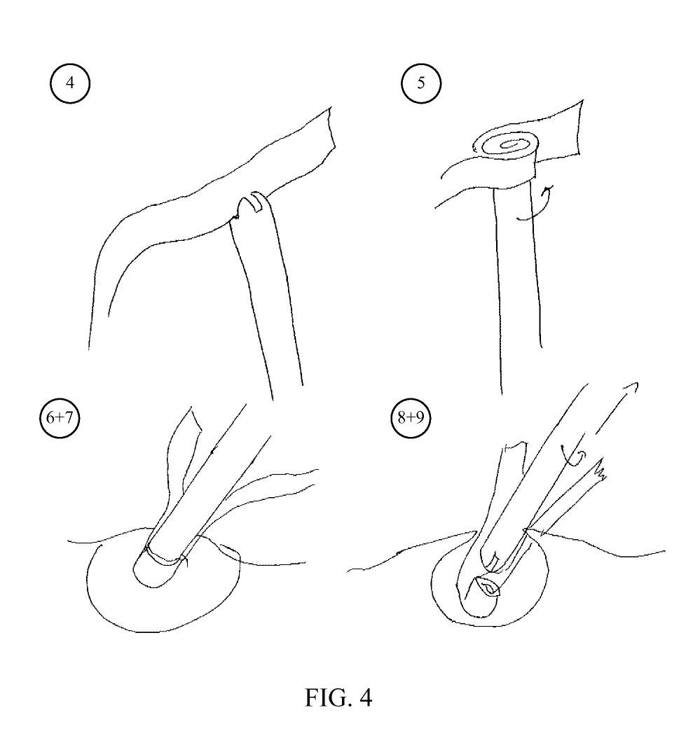

As can be seen in FIG. 4, the present invention is then used to guide the packing strip into the sub-dermal cavity of the abscess, the ulcer, or the deep wound. To being this portion of the process, the packing strip is inserted into a particular slot from the plurality of slots 6 so that the packing strip can engage the probing end. Next, the handle portion 2 is rotated in a first direction until the packing strip wraps around the probing tip 4, which adequately secures the packing strip to the probing tip 4. The first direction can either be clockwise or counterclockwise. The process continues by pushing the packing strip into the abscess, the ulcer, or the deep wound with the probing tip 4, which fills the sub-dermal cavity with the packing strip. The user can then position the packing strip into the proper area within the sub-dermal cavity by guiding probing tip 4 with the handle portion 2. Once the packing strip is in the properly positioned, the handle portion 2 is rotated in a second direction in order to unwrap and disengage the packing strip from the probing tip 4. The second direction is opposite to the first direction and can be either counterclockwise or clockwise. The probing tip 4 is then removed from the abscess, the ulcer, or the deep wound so that the user can repeat the aforementioned steps to guide the remainder of the packing strip into the sub-dermal cavity.

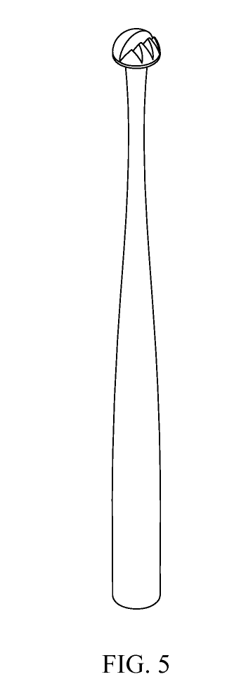

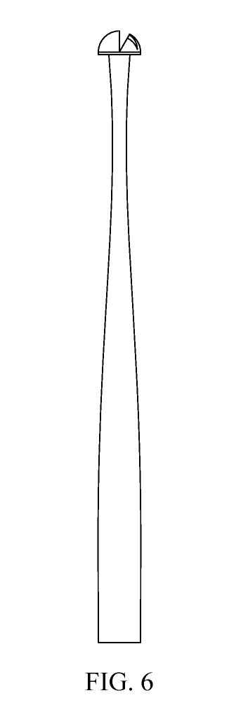

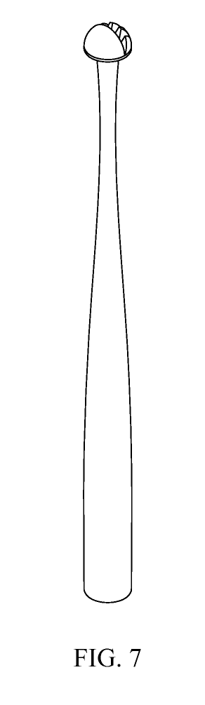

A different embodiment of the present invention allows the user to complete the same process of medically treating a sub-dermal cavity left by an abscess, an ulcer, or a deep wound. The different embodiment of the present invention is shown in FIGS. 5, 6, and 7, which illustrates the orientation of the components. The primary difference between this embodiment and the preferred embodiment is that the blunt tip 3 and the probing tip 4 are combined onto one end of the rod 1.

Although the invention has been explained in relation to its preferred embodiment, it is to be understood that many other possible modifications and variations can be made without departing from the spirit and scope of the invention as hereinafter claimed.

What is claimed is:

1. A packing strip probe comprises,
a rod;
a blunt tip;
a probing tip;
a rod comprises a handle portion;
a probing tip comprises a plurality of tines, a plurality of slots, and a rounded end;
said blunt tip being located opposite of said probing tip on said rod;
said handle portion being located in between said blunt tip and said probing tip;
said plurality of slots traversing into said rod and laterally traversing through said rod;
said plurality of tines being shaped by said plurality of slots; and
said rounded end being formed on said plurality of tines opposite of said rod.

2. The packing strip probe as claimed in claim 1 comprises,
said plurality of slots comprises a first slot and a second slot; and
said first slot being perpendicularly positioned to said second slot.

3. A method of medically treating an abscess, an ulcer, or a deep wound with a packing strip probe, the method comprises the steps of:
providing a rod with a handle portion, a blunt tip, and a probing tip, wherein said probing tip comprises a rounded end, a plurality of tines, and a plurality of slots;
providing a packing strip, wherein said packing strip is used to fill a sub-dermal cavity left by an abscess, an ulcer, or a deep wound;
(1) forcing said blunt end into said abscess, said ulcer, or said deep wound in order to open up said abscess, said ulcer, or said deep wound;
(2) gently debriding said abscess, said ulcer, or said deep wound with said blunt tip;
(3) aggressively debriding said abscess, said ulcer, or said deep wound with said rounded end and said plurality of tines on said probing tip;
(4) inserting said packing strip into a particular slot from said plurality of slots in order to engage said packing strip to said probing end;
(5) rotating said handle portion in a first direction in order to wrap said packing strip around said probing tip;
(6) pushing said packing strip into said abscess, said ulcer, or said deep wound with said probing tip in order to fill said sub-dermal cavity left by said abscess, said ulcer, or said deep wound with said packing strip;
(7) positioning said packing strip within said dermal cavity by guiding said probing tip with said handle portion;
(8) rotating said handle portion in a second direction in order to unwrap and disengage said packing strip from said probing tip, wherein said second direction is opposite of said first direction;
(9) pulling said probing tip out of said abscess, said ulcer, or said deep wound; and
(10) repeating steps (4) through (9) in order to guide remainder of said packing strip into said dermal cavity.

4. A packing strip probe comprises, a rod;
a blunt tip;
a probing tip;
a rod comprises a handle portion;
a probing tip comprises a plurality of tines, a plurality of slots, and a rounded end;
said blunt tip being located opposite of said probing tip on said rod;
said handle portion being located in between said blunt tip and said probing tip;
said plurality of slots traversing into said rod and laterally traversing through said rod;
said plurality of tines being shaped by said plurality of slots;
said rounded end being formed on said plurality of tines opposite of said rod;
said plurality of slots comprises a first slot and a second slot; and
said first slot being perpendicularly positioned to said second slot.
